(19) United States
(12) Patent Application Publication
(10) Pub. No.: US 2018/0310986 A1
(43) Pub. Date: Nov. 1, 2018

(54) HIGH SURFACE ENERGY PORTION ON A MEDICAL INSTRUMENT

(71) Applicant: GYRUS ACMI, INC., d/b/a Olympus Surgical Technologies America, Southborough, MA (US)

(72) Inventors: Kester J. Batchelor, Mound, MN (US); Theodore C. Blus, Shoreview, MN (US); Huisun Wang, N Maple Grove, MN (US); Riyad Moe, Madison, WI (US)

(21) Appl. No.: 15/772,624

(22) PCT Filed: Dec. 21, 2015
(86) PCT No.: PCT/US15/67015

§ 371 Date: May 1, 2018

(51) Int. Cl. A61B 18/14

(52) U.S. Cl. CPC A61B 18/1445; A61B 2017/00858

(57) ABSTRACT

Abstract: A medical instrument includes an end effector and one or more areas of high surface energy on at least a portion of the end effector. The one or more areas of high surface energy are configured to engage and draw away an object or anatomical feature.

### FIELD

These teachings relate generally to medical instruments, and more particularly to an end effector having one or more areas of high surface energy for manipulating or otherwise effecting an object or feature of fee anatomy.

### BACKGROUND

Some medical instruments have one or more end effectors for manipulating or otherwise effecting an object or a feature of interest of the anatomy. For example, some medical forceps, probes, or spatulas include an end effector for moving, gripping, grasping, pushing, pulling, cutting, coagulating, dissecting, and/or otherwise effecting a vessel or tissue during a medical procedure.

Some medical instruments have an end effector that is relatively smooth or has low-friction so that the end effector can easily pass through tissue planes. However, while manipulating or otherwise effecting a vessel or tissue, a smooth or low-friction end effector may unintentionally allow the vessel or tissue to slip from the end effector, which may damage or otherwise cause trauma to the vessel or tissue; may unnecessarily prolong a medical procedure; and/or may be cumbersome for the surgeon performing the procedure. Some medical instruments have an end effector with ridges or teeth aimed at preventing a vessel or tissue from slipping from the end effector; however, the ridges or teeth may damage or otherwise cause trauma, to a vessel or tissue, especially if the vessel or tissue is fragile or already inflamed or damaged.

Some examples of medical instruments and end effectors are disclosed in U.S. Pat. Nos. 4,958.539; 5,658,307; 7,204,835; 8,262,655; and 8,968,358—the disclosures of which are all hereby incorporated by reference herein for all purposes.

It may be desirable to improve the current state of the art by proving a medical instrument and/or an end effector that can easily pass through tissue planes while also preventing a vessel or tissue from slipping from the end effector. It may be desirable to provide an end effector that can prevent an object or anatomical feature such as a vessel or tissue, for example, from slipping therefrom without causing trauma or damage to the object, vessel, or tissue.

### SUMMARY

The teachings included herein provide a medical instrument and/or an end effector for use in open or laparoscopic procedures. The medical instrument, the end effector, or both includes one or areas having high surface energy. The end effector also includes one or more areas that are free of the high surface energy. The areas of high surface energy may function to help move or manipulate an object or anatomical feature, such as a vessel or tissue, without the object or anatomical feature slipping from the end effector. The one or more areas that are tree of the high surface energy may function to allow the end effector to easily pass through tissue planes without damaging or causing trauma to the tissue.

The present teachings also provide a medical instrument comprising an end effector and one or more areas of high surface energy on at least a portion of the end effector. The one or more areas of high surface energy are configured to engage and draw away tissue.

The present teachings further provide a forceps comprising a jaw assembly. The jaw assembly includes a first body having a top surface and a second body having a bottom surface. The top surface of the first body, the bottom surface of the second body, or both include one or more areas of high surface energy comprising silicone.

Further yet, the present teachings further provide a medical instrument comprising an end effector having a body and one or more areas of high surface energy on at least a portion of the body. The one or more areas of high surface energy are configured to engage and draw away tissue. The fine or more areas of high surface energy comprise silicone.

### BRIEF DESCRIPTION OF THE DRAWINGS

FIG. 1 is a perspective view of an end effector.

FIG. 2 is a perspective view of an end effector.

FIG. 3 is a perspective view of an end effector.

FIG. 4 is a perspective view of an end effector.

FIG. 5 is a perspective view of an end effector.

FIG. 6A is a top view of an end effector.

FIG. 6B is a side view of an end effector.

FIG. 7 is a side view of a medial instrument including an end effector.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which ore also hereby incorporated by reference into this written description.

The present teachings provide one or more medical instruments. The medical instrument can be any instrument suitable for manipulating, engaging, moving, grasping, gripping, constricting, pushing, pulling, cutting, tearing, coagulating, sealing, cauterizing, dissecting, fulgurating, or otherwise effecting an object or anatomical feature of interest. The anatomical feature of interest may be any anatomical feature, such as a vessel, tissue, vein, artery, the like, or a combination thereof. The medical instrument can be used in open procedures, laparoscopic procedures, or both. Exemplary medical instrument may include, but are not limited to, a forceps, a dissector, a dissector probe, a bulbous probe, scissors, a scalpel, a spatula, a J-hook, the like, or a combination thereof.

The medical instrument can be used with or without power. When used with power, the medical instrument can be used in electrosurgery. When used with power, one or more electrical currents, therapies, or signals may be provided to the medical instrument, the end effector, one or more electrodes, or a combination thereof. The medical instrument may be used with monopolar energy, bipolar energy, blended energy, or a combination thereof. The medical instrument can be used in a monopolar circuit, a bipolar circuit, or both. During use, a suitable current, therapy, and/or signal may be passed from, through, or between tire end effector, one or more electrodes, one or more bodies, a remote pad, a patient or anatomy, or a combination thereof so that an object or anatomical feature can be electrosurgically effected. For example, the object or anatomical feature can be cut, coagulated, welded, sealed, dissected, fulgurated, or otherwise effected, or a combination thereof.

The medical instrument may include a hand piece. The hand piece may function to house, support, and/or contain the end effector, one or more working functions or assemblies of the medical instrument, the parts of components needed to move or actuate the end effector or the one or more working functions or assemblies, or a combination thereof. The one or more working functions or assemblies may include a gripping function for gripping, pulling, constricting, coagulating, and/or sealing an object or anatomical feature; a cutting function for cutting, transecting, dissecting an object or anatomical feature; or a combination thereof. The hand piece may include sufficient controls for operating, actuating, and/or manipulating the end effector, the one or more working functions or assemblies of the effector or the medical instrument, or a combination thereof. The controls may be located anywhere on medical instrument, the hand piece, at a remote location, or a combination thereof. The hand piece may function to be held and/or manipulated by an operator or surgeon using one hand or both hands. The hand piece may function to be held and/or manipulated by an operator or surgeon and/or one or more assistants.

The medical instrument may include one or more mechanisms. The one or more mechanisms may function to manipulate, actuate or otherwise move or operate the one or more end effectors, working functions or assemblies of the end effector or medical instrument, or a combination thereof. For example, the one or more mechanisms may function to move, rotate, reciprocate, actuate, open, and/or close the end effector. If the end effector is a jaw assembly, the one or more mechanisms may function to move the jaw assembly between a closed or gripping position and an open position. In the closed position, the end effector, the jaw assembly, the one or more bodies, or a combination thereof may cooperate to manipulate, grip, grasp, and/or secure an object or anatomic feature in the jaw assembly or between the one or more bodies. When moving the jaw assembly or end effector into the closed position, using the hand piece or a mechanism therein as a reference, only the first body may be moved or pivoted towards the second body, only the second body may be moved or pivoted towards the first body, or both bodies may move or pivot towards one another. The first body, the second body, or both may move or pivot about a pivot so that the end effector can be used in an open position, a closed position, or both. When moving the jaw assembly or end effector into the closed position, using the hand piece or a mechanism therein as a reference, only the first body may be moved or pivoted towards the second body, only the second body may be moved or pivoted towards the first body, or both bodies may be moved or pivoted towards one another. In the open position, the one or more bodies are in a spaced apart relationship relative to one another. If the end effector includes a cutting element, the one or more mechanisms may function to move, reciprocate, and/or rotate the cutting element. The one or more mechanisms may be moved or actuated by moving or actuating one or more user inputs, such as one or more triggers, wheels, levers, buttons, knobs, the like, or a combination thereof located on the medical instrument, the hand piece, and/or at a remote location.

The medical instrument may include one or more elongated members. The elongated member may function to permit a portion of the medical instrument, such as the end effector, to be inserted into or extend into a patient or the anatomy, while a portion of the medical instrument, such as the hand piece, remains outside of the patient or anatomy. The elongated member may house or protect at least a portion of the end effector when the end effector is in an extended position, a retracted position, or both. The elongated member may extend along a longitudinal axis between a proximal end and a distal end. The distal end may extend with the end effector towards or into a patient, while a proximal end may engage the hand piece, the medical instrument or both. The one or more elongated members may be made from a material that is rigid, flexible, resilient, or a combination thereof. The one or more elongated members may be generally straight or linear, or may include one or more curves, bends, and/or arcs. The one or more elongated members may be any structure that may be moved, articulated and/or rotated by manipulating one or more of the user inputs, mechanisms, or both. The one or more elongated members may function to house, contain, actuate, move, retract, expand, and/or protect the one or more of the working functions or assemblies, the end effector, or a combination thereof. The one or more elongated members may be generally hollow and receive at least a portion of the end effector therein so that the end effector can be moved, reciprocated, rotated, etc. therein. The one or more elongated members may be generally solid end effector may be connected at or near a distal end thereof.

The medical instrument may include one or more end effectors. The one or more end effectors may function to manipulate, engage, move, grasp, grip, push, puff, cut, tear, coagulate, seal, cauterize, dissect, fulgurate, or otherwise effect an object or anatomical feature of interest, such as a vessel tissue, vein, artery, the like, or a combination thereof. The end effector may be removably connected to the medical instrument, the elongated member, or both so that the end effector can be easily separated from the medical instrument. The end effector may be fixedly connected to the medical instrument so that the end effector is not easily separable from the medical instrument. The end effector may be moveably connected to the medical instrument, the elongated member, the hand piece, or a combination thereof, so that the end effector can be moved, extended, retracted, articulated, rotated, opened, closed, or a combination thereof. The end effector may be, or may include, one or more jaw assemblies, one or more bodies, one or more cutting elements, one or more spatulas, one or more j-hooks, one or more probes, one or more bulbous probes, or a combination thereof.

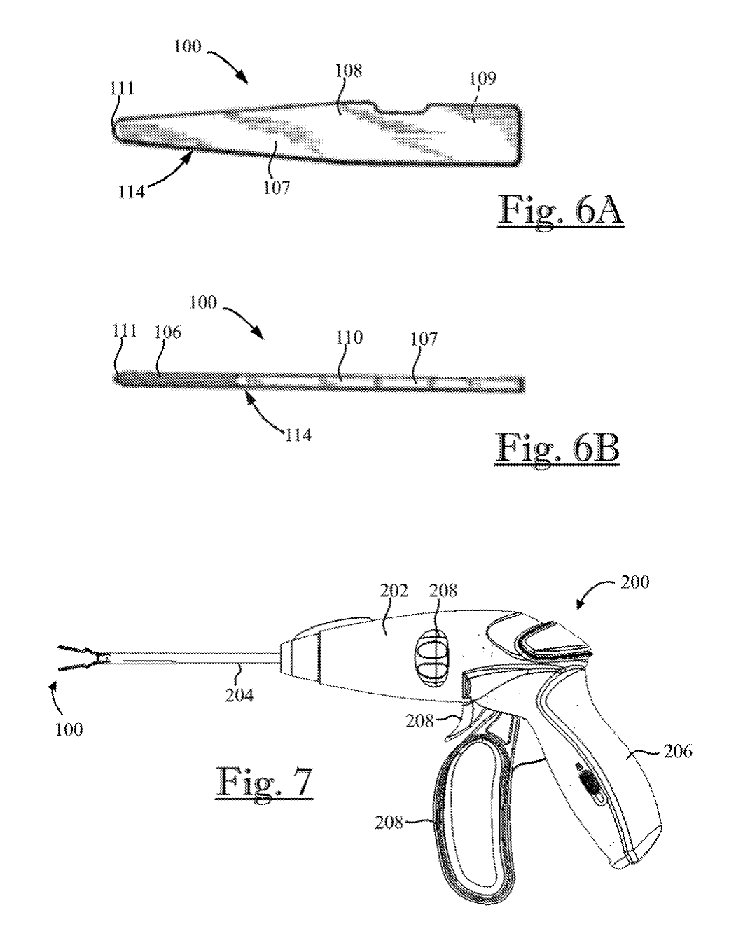

The end effector may be, or may include one or more bodies. If the end effector includes two or more bodies, one or more of the bodies may be moveable relative to one another. The end effector or the one or more bodies may be used for tissue dissection. The end effector or the one or more bodies may be used in one or more tissue dissection techniques, such as blunt dissection, lift dissection, spread dissection, and/or sweep dissection. In blunt dissection, an object or anatomical feature, such as a vessel or tissue, can be separated with a blunt object, or area of the end effector or the one or more bodies. In blunt dissection, the object or anatomical feature can be lifted, moved, separated, or repositioned with a top surface, a bottom surface, an inner or gripping surface, one or more side surfaces, one or more front surfaces, a distal end, or a combination thereof of the end effector and/or the one or more bodies. In lift dissection, the end effector or the bodies may be initially in either the open or closed position and then at feast one of the bodies may be moved to lift, move, and/or reposition a vessel or tissue. In lift dissection, depending on the position of end effector or bodies, a top surface, bottom surface, side surfaces, front surfaces, or an inner surface may be used to lift, move, and/or reposition the vessel or tissue. In spread dissection, the end effector or bodies may be placed into an area, such as an intended dissection plane, while in the closed position and then moved into an open position. Moving the end effector or bodies into the open position may cause the top surface, the bottom surface, the side surfaces, front surfaces, or a combination thereof to spread, move, and/or reposition a vessel or tissue. In sweep dissection, the end effector may be in either the closed or open position, and a top surface, a bottom surface, a front surface, or side surfaces of one or both of the bodies may be brushed, moved or “swept” across a vessel or tissue to move, reconfigure, and/or reposition vessel or tissue. If the end effector is a non-jawed probe, for example as illustrated in FIGS. 5, 6A, and/or 6B, the end effector or body may be brushed, moved or “swept” across the vessel or tissue to move, reconfigure, and/or reposition a vessel or tissue.

The end effector or the one or more bodies may comprise a first body and a second body. The first body may oppose the second body. The first body may be an upper body and the second bods may be a lower body, or vice versa depending on an orientation of the end effector, the medical instrument, a surgical site, or a combination thereof. One or both of the bodies may be moved or may be pivoted towards the other to create a gripping force, to grip or hold an object or feature of interest, to move the bodies into a closed or gripping position, or a combination thereof. One or both of the bodies may be moved or pivoted away from the other to release a gripping force, to release an object or feature of interest, to move the bodies into a open position, or a combination thereof. One or both of the bodies may be moved or pivoted towards or away from the other. For example, relative to the hand piece or a mechanism, the first body may be moved or pivoted towards or away from the second body, or vice versa. For example, relative to the hand piece or a mechanism, both the first body and the second body may move or pivot towards or away from one another. The end effector may be configured to interface with and dissect tissue during movement of the first body and the second body from the closed position to the open position in a plane of motion defined by the first body and the second body such that the one or more areas of high surface energy define a leading edge that is configured to interface with the tissue that is within the plane of motion.

The end effector or the one or more bodies may be made of a material that is at least partially flexible, resilient, rigid, deformable, or a combination thereof. The end effector or the one or more bodies may be made of a bulk conductor, an insulator, an electrode, or a combination thereof. In some configurations, the end effector or the one or more bodies may be formed from a conductor, and may include an insulating shell or skin disposed on or over at least a portion of the outer surface, the top surface, the bottom surface, the side surfaces, the front surface, or a combination thereof. The end effector or the one or more bodies may be connected to a generator via one or more wires or leads so that electricity, one or more therapy currents, or a combination thereof can be applied to an object or feature of interest via the one or more bodies, electrodes, cutting elements, or a combination thereof.

The end effector or the one or more bodies may have any suitable shape. For example, the end effector and/or the one or more bodies may have a generally cubic shape or cross section, a rectangular shape or cross section, an ovoid shape or cross section, an elongated shape or cross section, or any other suitable shape or cross section for use in medical procedures. The end effector and/or the one or more bodies may have a width that is longer, shorter, or the same size as a length, and a thickness that is larger, smaller, or the same size as the length and/or the width. The length may extend between a proximal and distal end of the effector, body, or both, and the width may be generally transverse to the length. The thickness may be generally transverse to both the length and the width. For example, the one or more bodies may have a shape suitable for forming or being one or more jaws or bodies of a jaw assembly; a spatula; a J-hook; a probe; a bulbous probes; the like, or a combination thereof. The one or more bodies may include a top surface, a bottom surface, or both. The top surface, the bottom surface, or both may function to engage and/or otherwise effect an object or anatomical feature. For example, the top surface, the bottom surface, or both may engage an object or anatomical feature to move, push, pull, draw away, effect, dissect, coagulate, weld, seal, and/or otherwise effect an object or anatomical feature. The top surface and/or the bottom surface may be located generally opposite an inner or gripping surface. The top surface and/or the bottom surface may be located generally perpendicular to one or more side surfaces, a front surface, or both. The top surface, the bottom surface, or both may include one or more areas of high surface energy, or may include one or more areas that are free of the high surface energy. The top surface, the bottom surface, or a combination thereof may be substantially smooth and free of any projections, teeth, protuberances, nubs, bumps, gripping surfaces, high surface energy portions, or a combination thereof. The top surface, the bottom surface, or both may include projections, teeth, protuberances, nubs, bumps, gripping surfaces, high surface energy portions, or a combination thereof.

The end effector or the one or more bodies may include one or more side surfaces. The one or more side surfaces may function to engage and/or otherwise effect an object or anatomical feature. For example, the one or more side surfaces may engage an object or anatomical feature to move, push, pull, draw away, dissect, coagulate, weld, seal, and/or otherwise effect an object or anatomical feature. The one or more side surfaces may be located generally perpendicular to top surface, the bottom surface, or both. The one or more side surfaces may include one or more areas of high surface energy, or may include one or more areas that are free of the high surface energy. The one or more side surfaces may be substantially smooth and free of any projections, teeth, protuberances, nubs, bumps, gripping surfaces, high surface energy portions, or a combination thereof. The one or more side surfaces may have a coefficient of friction that is less than, the same as, or greater than the coefficient of friction of the areas with high surface energy, the top surface, the bottom surface, the front surface, or a combination thereof. The one or more side surfaces may include projections, teeth, protuberances, nubs, bumps, gripping surfaces, high surface energy portions, or a combination thereof.

The end effector or the one or more bodies may include a front surface. The front surface may function to engage and/or otherwise effect an object or anatomical feature. For example, the front surface may engage an object or anatomical feature to move, push, pull, draw away, dissect, and/or otherwise effect an object or anatomical feature. The front surface may function to be used for blunt dissection. The front surface may be a nose portion, a distal end or tip of the effector, the one or more bodies, of both. The front surface may be located generally perpendicular to top surface, the bottom surface, or both. The front surface may be generally flat and/or straight, or the front surface may be tapered. The front surface may include one or more areas of high surface energy, or may include one or more areas that are free of the high surface energy. The front surface may be substantially smooth and free of any projections, teeth, protuberances, nubs, bumps, gripping surfaces, high surface energy portions, or a combination thereof. The front surface may have a coefficient of friction that is less than, the same, or greater than the coefficient of friction of the areas with high surface energy, the top surface, the bottom surface, the side surfaces, or a combination thereof. The front surface may include projections, teeth, protuberances, nubs, bumps, gripping surfaces, high surface energy portions, or a combination thereof.

The effector or the one or more of the bodies may include one or more inner or gripping surfaces. The one or more inner or gripping surfaces may function for gripping, holding, manipulating, pulling, and/or otherwise effecting an object or feature of interest of the anatomy. The one or more inner or gripping surface may be located on a body opposite a top surface, a bottom surface, or both. The one or more inner or gripping surfaces may be at least partially smooth, flat, contoured, serrated, textured, toothed, undulating, wave-shaped, planar, irregular, knurled, grit blasted, or a combination thereof. The one or more gripping surfaces may include one or more surfaces that are horizontal, vertical, canted, or a combination thereof relative to a longitudinal axis extending from a proximal end of the effector to a distal end of the effector. The one or more inner or gripping surfaces may include one or more ridges, teeth, mouse teeth, gaps, openings, of a combination thereof. The one or more inner or gripping surfaces may include one or more electrodes in communication with a generator for electro-surgically cutting and/or coagulating an object or anatomical feature. The one or more inner or gripping surfaces may be include one or more areas of high surface energy, or may include one or more areas that are free of the high surface energy. The one or more inner or gripping surfaces may be any surface that comes into contact with the anatomy. The one or more inner or gripping surfaces may be located opposite the one or more outer surfaces, side surfaces, or both.

The end effector, the one or more bodies, or both may include one or more areas or portions of high surface energy. The one or more areas or portions of high surface energy may function to effectively manipulate, engage, move, grasp, grip push, pull, draw away, cut, tear, dissect, or otherwise effect an object or anatomical feature of interest, such as a vessel, tissue, vein, artery, the like, or a combination thereof. The one or more areas of high surface energy may function to dissect tissue along a tissue plane, facilitate separation of tissue, or both. The one or more areas of high surface energy may optimize the end effector, jaw assembly, or both for tissue lift or lift dissection; tissue spread or spread dissection; tissue sweep or sweep dissection, or a combination thereof. The one or more areas of high surface energy may function to frictionally engage an object or anatomical feature of interest without causing trauma, perforating, or otherwise damaging the feature of interest, especially if the feature of interest is already damaged, inflamed, and/or infected. This may be especially desirable when dissecting or otherwise effecting a thin and/or fragile anatomical feature of interest. The one or more areas of high surface energy may function to prevent slipping of an object or anatomical feature from the end effector, jaw assembly, the one or more bodies, or a combination thereof especially when the end effector is moved, rotated, pivoted, opened, closed, or otherwise manipulated.

In electrosurgical applications, the one or more areas of high surface energy may function to insulate heat, electricity, current, or a combination thereof. In this regard, a feature of interest contacted by the end effector, one or more of the bodies, the one or more areas of high surface energy, the end effector, or a combination thereof is prevented from burning, heating, charring, or a combination thereof.

The one or more areas of high surface energy can be located in one or more areas or locations on the medical instrument, the end effector, the jaw assembly, the bodies, or a combination thereof. For example, the one or snore areas of high surface energy can be located at a distal end (i.e., a nose portion or front surface) of the end effector, jaw assembly, probe, spatula, the one or more bodies, or a combination thereof. The one or more areas of high surface energy can be located on a top or upper surface, a bottom or lower surface, an inner or gripping surface, or a combination thereof. The one or more side surfaces of the end effector, the bodies, the probe, the spatula, and/or the jaw assembly may include one or more areas of high surface energy. However, it may be desirable for one or more of the side surfaces to be free of the high surface energy areas or portions, nubs, protrusions, projections, or a combination thereof so that the end effector, jaw assembly, bodies, probe, spatula, or a combination thereof can pass through tissue planes with lower resistance or drag.

The one or more areas of high surface energy can be any suitable size, shape or geometry. For example, the one or more areas of high surface energy can have a shape that compliments the top and/or bottom surfaces of the end effector, jaw assembly, probe, bodies, spatula, or a combination thereof; the side surfaces; and/or a proximal and/or distal end of the end effector, jaw assembly, bodies, or a combination thereof. The one or more areas of high surface energy can be a ridge or pad. The one or more areas of high surface energy can be an area, ridge, or pad that is wider across a width of the body, end effector, probe, spatula, and/or jaw assembly and narrower across a length of the body, end effector and/or jaw assembly, or vice versa. The length may extend in a direction generally along a long length of the effector, for example between a proximal and distal end of the body. The width may be generally transverse to the length of the body. The body also includes a thickness that is generally transverse to the length and the width thereof. The one or more areas of high surface energy can have a proximal section that is wider than a distal section, or vice versa. Accordingly, the one or more areas of high surface energy can include one or more tapered portions. The one or more areas of high surface energy can be one or more localized regions or dots on the medical instrument or jaw assembly.

The one or more areas of high surface energy can be, or may include, one or more disruptions, nubs, bumps, ribs, or dot sections; and/or may include one or more textured, modified, or disrupted surfaces. The one or more areas of high surface energy can have one or more protuberances, teeth, peaks, troughs, indentations, undulations, and/or projections. The one or more areas of high surface energy can be substantially free of one or more disruptions, nubs, bumps, ribs, or dot sections; substantially free of one or more textured, modified, or disrupted surfaces; and/or substantially free of one or more. The one or more areas of high surface energy can be substantially free of protuberances, teeth, peaks, troughs, indentations, undulations, and/or projections. The one or more areas of high surface energy can be one or more abrasive areas. The one or more areas of high surface energy can be substantially smooth and free of abrasive areas. The one or more areas of high surface energy may be sticky or tacky to the touch, or may be free from being sticky or tacky to the touch. The one or more areas of high surface energy or nubs may be located on the same plane as the underlying surface of the body. That is, the one or more areas of high surface energy or nabs may be free from extending, or may not extend, above or proud of the underlying surface. Instead, the one or more areas of high surface energy or nubs may be substantially smooth and/or parallel relative to the underlying surface. The one or more areas of high surface energy can be located on the same plane as the underlying surface. The one or more areas of high surface energy or nubs may extend above or proud of the underlying surface or a plane of the underlying surface.

The one or more areas of high surface energy can be a coating applied to the jaw assembly, end effector and/or the one or more bodies. The one or more high surface energy portions may be located on an insulating cover or layer on the one or more bodies, jaw assembly, and/or end effector. The one or more areas of high surface energy can be printed onto the end effector or the one or more bodies, jaw assembly, and/or end effector. The one or more areas of high surface energy can be insert molded onto the jaw assembly, probe, spatula, end effector and/or the one or more bodies. The one or more areas of high surface energy can be over molded, insert molded, or molded onto the end effector or the one or more bodies. The one or more areas of high surface energy can be integrally formed with the jaw assembly, end effector and/or the one or more bodies. The one or more areas of high surface energy can be mechanically attached with suitable fasteners, bonded, or adhered to or onto the end effector or to the one or more bodies. The one or more areas of high surface energy can fee one or more ground, cut, or machined areas; sand or glass blasted areas; chemically or laser etched areas; hatched areas; knurled areas; sprayed areas; abraded areas, or a combination thereof. The one or more areas of high surface energy can be integrally formed with an insulator located on the end effector and/or the one or more bodies.

The one or more areas of high surface energy can comprise one or more suitable materials such as silicone, rubber, silicone rubber, tungsten carbide, ceramic, ceramic powders, fabrics, nickel, and/or electrodeless nickel coatings. The one or more areas of high surface energy may formed from a material that is soft to the touch, hard to the touch, compliant, or a combination thereof. The one or more areas of high surface energy may formed from a material that is sticky or tacky to the touch. The one or more areas of high surface energy can be made from the same material of the end effector. The one or more areas of high surface energy can be formed in the same process when the end effector is formed, or in a subsequent process. The one or more areas of high surface energy can be part of or function as one or more thermal or electrical insulators. For example, the one or more areas of high surface energy can be part of or incorporated into a material or feature that thermally and/or electrically insulates an electrode, a blade, a blade electrode, a working arm, a cutting element, or a combination thereof from one or both of the bodies, the end effector, the jaw assembly, or a combination thereof. For example, the thermal and/or electrical insulator material or feature may extend from an inner or gripping surface to, through, or around the outer, top, bottom, lower, or side surfaces. For example, the thermal and/or electrical insulator material or feature may insulate a center electrode, blade electrode, cutting blade, etc. from a lateral jaw electrode, one or both of the bodies that may be electrically connected to a generator, or a combination thereof.

The one or more areas of high surface energy may have a coefficient of friction that is 0.1 or greater, 0.2 or greater, 0.3 or greater, 0.4 or greater. 0.5 or greater, preferably, 0.6 or greater, 0.7 or greater, 0.8 or greater, 0.9 or greater. The one or more areas of high surface energy may have a coefficient of friction that is 0.3 or less, 0.8 or less, or even 0.7 or less, 0.6 or less, 0.5 or less, 0.4 or less, 0.3 or less, 0.2 or less, or even 0.1 or less. The one or more areas of high surface energy may have a coefficient of friction that is common or the same across the one or more areas, or the coefficient of friction can vary or change amongst the different areas. For example, a distal portion of an area of high surface energy may have a greater coefficient of friction than a proximal area, or vice versa. Similarly, a medial area may have a greater coefficient of friction than lateral portions, or vice versa. Preferably, the coefficient of friction is large enough to move an object or anatomical feature, or create a tissue plane, but not large enough to cause trauma to an object or anatomical feature.

The one or more effectors, jaws, bodies, areas of high surface energy, or a combination thereof can be exposed to one or more sterilization cycles without degrading or otherwise failing. The one or more effectors, jaws, bodies, areas of high surface energy, or a combination thereof can be exposed to one or more sterilization cycles without effecting the tackiness, stickiness and/or the coefficient of friction thereof. Exposure to one or more sterilization cycles may permit for the one or more effectors, bodies, areas of high surface energy, or a combination thereof to be reused. A sterilization cycle may include subjecting the medical instrument, the end effector, the one or more bodies, etc. to heating, cooling, and/or exposure to one or more sterilizing mediums. The one or more areas of high surface energy may withstand 10 or more sterilization cycles, 20 or more sterilization cycles, 50 or more sterilization cycles, 40 or more sterilization cycles, or, preferably 50 or more sterilization cycles. The one or more areas of high surface energy can withstand 100 or less sterilization cycles, 80 or less sterilization cycles, 60 or less sterilization cycles, or 55 or less sterilization cycles. The one or more areas of high surface energy can withstand heat and/or exposure to one or more therapy currents used in electro surgery.

The end effector, the one or more bodies, or both may include one or more areas that are free of high surface energy. That is, the end effector, the one or more bodies, or both may include one or more areas where the high surface energy does not exist; one or more areas where the coefficient of friction is lower than the coefficient of friction in the areas where the high surface energy exits, or a combination thereof. For example, the one or more areas that are free of high surface energy may have a coefficient of friction that is 0.9 or less, 0.8 g or less, 0.7 or less, 0.6 or less, 0.5 or less, 0.4 or less, 0.3 or less, 0.2 or less, or even 0.1 or less. The one or more areas that are free of high surface energy may have a coefficient of friction that is 0.1 or greater, 0.2 or greater, 0.3 or greater, 0.4 or greater, 0.5 or greater, 0.6 or greater, 0.7 or greater, 0.8 or greater, 0.9 or greater. The one or more areas that are free of high surface energy may function to effectively separate tissue along a tissue plane; facilitate separation of tissue, or both. The one or more areas that are free of high surface energy may function to allow the end effector, jaw assembly, bodies, probe, spatula, or a combination thereof to easily pass through tissue planes with little to no restriction or drag. The one or more areas that are free of high surface energy may be free of any material other than the material comprising the end effector, the bodies, or both. The one or more areas that are tree of high surface energy may include one or more suitable materials covering the end effector, bodies, or both, that has a coefficient of friction that is lower than the high surface energy areas. The one or more areas of high surface energy may have a coefficient of friction that is greater than the one or more areas that are free of high surface energy by 0.1 times or more, 0.5 limes or more, 1 time or more, 2 times or more, 4 times or more, 5 times or more, 7 times or more, or even 9 times or more. The one or more areas of high surface energy may have a coefficient of friction that is less than the one or more areas that are free of high surface energy by 10 times or less, 7 times or less, 5 times or less, 3 times or less, 2 times or less, 1 time or less, 0.5 times or less. The one or more areas that are free of the high surface energy may be located on the front surface, top surface, upper surface, bottom surface, lower surface, front surface, and/or side surfaces of the first body, the second body, or both.

The medical instrument, the end effector, the jaw assembly, or a combination thereof may include a cutting element. The cutting element may function to cut or dissect an object, an anatomical feature, or both. The cutting element may be an electrode, a cutting blade, scalpel, or a combination thereof. The cutting element may be in communication with a generator so that the cutting element can be used in electrosurgery. The cutting element can be received in the jaw assembly, between the bodies, or both. The cutting element can be moved or reciprocated while the jaw assembly, the end effector, or both is in the closed position, the open position, or both. The cutting element can be electrically isolated, thermally isolated, or both from the one or more bodies, the one or more gripping portions, the jaw assembly, or a combination thereof via one or more insulating layers. The one or more insulating layers may include one or more areas having high surface energy, one or more areas that are free of high surface energy, or a combination of both. For example, the one or more insulating layers may be located on an inner or gripping surface of one of the bodies, and may extend through or around the body to an outer surface where the one or more areas of high surface energy may be located.

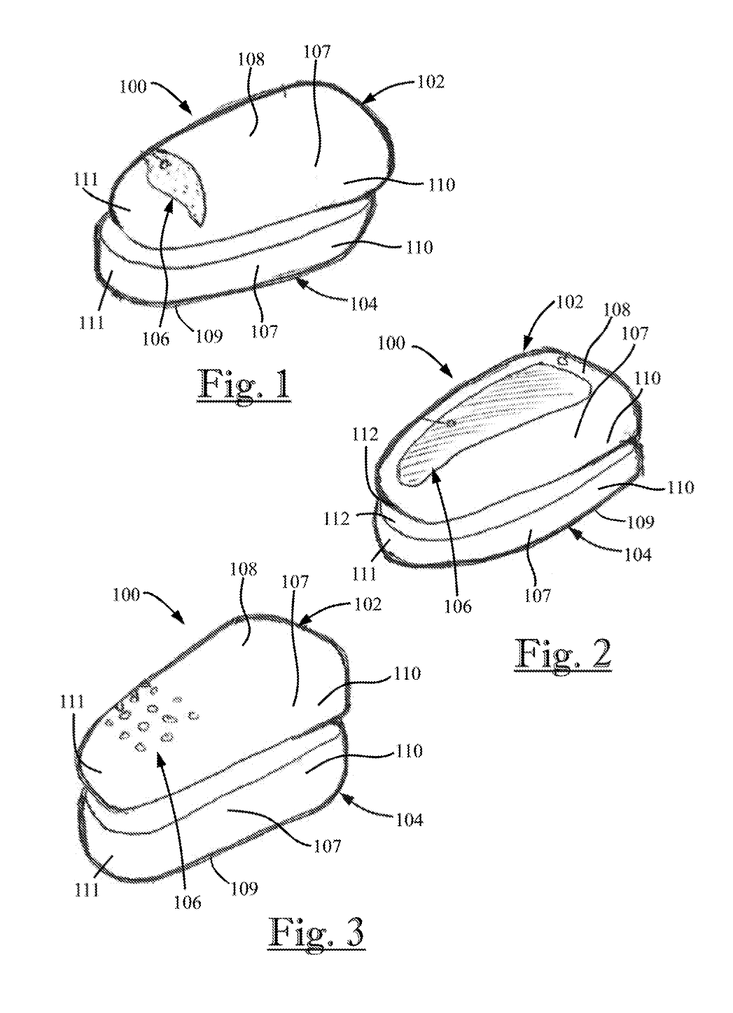

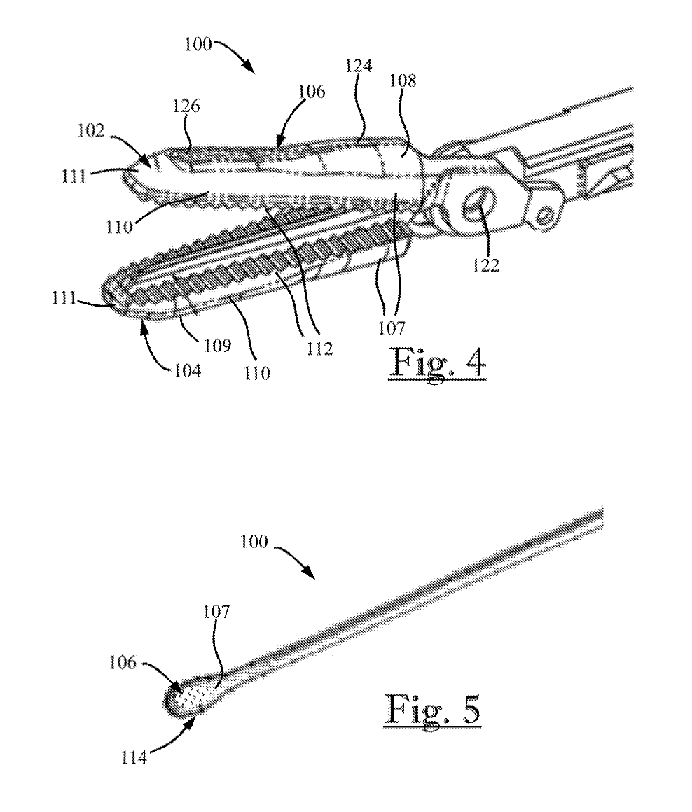

FIGS. 1-3 each illustrate an exemplary end effector 100. The end effector 100 is a jaw assembly illustrated in a closed position. The end effector 100 includes a first body 102 and a second body 104. The first body 102 has a top surface 108, side surfaces 110, and a front or distal surface 111, The second body 104 has a bottom surface 109, side surfaces 110, and a front or distal surface 111. The first body 102, the second body 104, or both may include an inner or gripping surface 112. Depending on the orientation of the end effector 100 and/or medical instrument 200, the top surface 108 may be the bottom surface 109 or vice versa, and the first body 102 may be the second body 104, or vice versa. The first body 102, the second body 104, or both include an area 106 of high surface energy, and areas 107 that are free of high surface energy. As illustrated in FIG. 4, the area 106 of high surface energy may include a portion where the coefficient of friction is the same, greater than, or less than the coefficient of friction at a another portion of the area 106.

FIG. 4 illustrates an exemplary end effector 100. The end effector 100 is a jaw assembly illustrated in an open position. The end effector 100 includes a first body 102 and a second body 104. The end effector includes a pivot 122 about which the first body 102, the second body 104 or both move or pivot so that the end effector can move between the open position and a closed position. The first body 102 has a top surface 108, side surfaces 110, and a front surface or distal surface 111. The second body 104 has a bottom surface 109, side surfaces 110, and a front surface or distal surface 111. The first body 102, the second, body 104, or both may include an inner or gripping surface 112. Depending on the orientation of the end effector 100 and/or medical instrument 200, the top surface 108 may be the bottom surface 109 or vice versa, and the first body 102 may be the second body 104 or vice versa. The first body 102, the second body 104, or both include an area 106 of high surface energy, and areas 107 that are free of high surface energy. The area 106 of high surface energy may include a first portion 124 and a second portion 126. The coefficient of friction of the first portion 124 may be the same, greater than, or less than the coefficient of friction at the second portion 126.

FIG. 5 illustrates an exemplary end effector 100. The end effector 100 is a probe. The end effector 100 includes a body 114. The body 114 includes an area 106 of high surface energy includes and areas 107 that are free of high surface energy.

FIG. 6A illustrates a top view of an exemplary end effector 100, and FIG. 6B illustrates a side view of an exemplary end effector 100. The end effector 100 illustrated an FIGS. 6A and 6B is a spatula. The end effector 100 includes a body 114. The body 114 includes a top surface 108, an opposing bottom surface 109, side surfaces 110, and a front or distal surface 111. Depending on the orientation of the end effector 100 and/or medical instrument 200, the top surface 108 may be the bottom surface 109 or vice versa. The body 114 includes an area 106 of high, surface energy and areas 107 that are tree of high surface energy. The area 106 of high surface energy may include a first portion where the coefficient of friction may be the same, greater than, or less than the coefficient of friction at a second portion of the area 106.

FIG. 7 illustrates a medical instrument 200. The medical instrument 200 includes an elongated member 204 extending from a hand piece 202. An end effector 100 extends from the elongated member 204. It is understood that the end effector 100 illustrated in FIG. 7 can be any end effector described and/or illustrated herein. The hand piece 202 includes a gripping portion 206 and one or more mechanisms 208 for manipulating the end effector 100, the elongated member 204, or both.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weigh. Thus, an expression in the Detailed Description of the Teachings of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

1) A medical instalment comprising:
i. an end effector; and
ii. one or more areas of high surface energy on at least a portion of the end effector,
wherein the one or more areas of high surface energy are configured to engage and draw away tissue, and
wherein the end effector comprises a first body, and the one or more areas of high surface energy are disposed on a top surface of the first body.

2) The medical instrument of claim 1, wherein the one or more areas of high surface energy comprise silicone.

3) (canceled)

4) The medical instrument of claim 1, wherein the end effector includes a second body, and
wherein the one or more areas of high surface energy are disposed on a bottom surface of the second body.

5) The medical instrument of claim 1, wherein the one or more areas of high surface energy are substantially smooth and free of protuberances.

6) The medical instrument of claim 1, wherein the first body includes one or more side surfaces, and
wherein the one or more side surfaces are substantially free of the one or more areas of high surface energy.

7) (canceled)

8) (canceled)

9) (canceled)

10) The medical instrument of claim 1, wherein the one or more areas of high surface energy have a coefficient of friction of about 0.6 or greater.

11) The medical instrument of claim 10, wherein the one or more areas of high surface energy has a first portion with a first coefficient of friction and a second portion with a second coefficient of friction, and
wherein the first coefficient of friction and second coefficient of friction are not equal.

12) (canceled)

13) The medical instrument of claim 1, wherein the one or more areas of high surface energy are tacky to the touch.

14) The medical instrument of claim 1, wherein wherein the one or more areas of high surface energy include one or more nubs.

15) The medical instrument of claim 1, wherein the one or more areas of high surface energy include a pad having a width at a proximal end of the pad that is wider than a width at a distal end of the pad.

16) The medical instrument of claim 1, wherein the one or more areas of high surface energy include an insert that is over molded onto the end effector.

17) The medical instrument of claim 1, wherein the one or more areas of high surface energy are integrally formed with the end effector.

18) The medical instrument of claim 1, wherein the one or more areas of high surface energy are integrally formed with an insulator located on the end effector.

19) (canceled)

20) The medical instrument of claim 1, wherein the one or more areas of high surface energy are adhered onto the end effector.

21) The medical instrument of claim 1, wherein the one or more areas of high surface energy can withstand at least one sterilization cycle.

22) (canceled)

23) (canceled)

24) The medical instrument of claim 4, wherein the first body, the second body, or both are pivotable between an open position and a closed position.

25) The medical instrument of claim 4, wherein the end effector is configured to interface with and dissect tissue during movement of the first body and the second body from a closed position to an open position in a plane of motion defined by the first body and the second body such that the one or more areas of high surface energy define a leading edge that is configured to interface with the tissue that is within the plane of motion.

26) (canceled)

27) (canceled)

28) A forceps comprising:
a jaw assembly including:
i. a first body having a top surface; and
ii. a second body having a bottom surface;
wherein the top surface of the first body, the bottom surface of the second body, or both include one or more areas of high surface energy comprising silicone.

29) The forceps of claim 28, wherein a side surface of the first body, a side surface of the second body, or both is free of the one or more areas of high surface energy.

30) (canceled)

31) (canceled)

32) (canceled)

33) (canceled)

34) (canceled)

35) (canceled)

36) (canceled)

37) (canceled)

38) (canceled)

39) (canceled)

40) (canceled)

41) The forceps of claim 28, wherein the jaw assembly is removeably connected to the forceps.
